(19) United States
(12) Patent Application Publication
(10) Pub. No.: US 2010/0167921 A1
(43) Pub. Date: Jul. 1, 2010

(54) Process for Roasting Diatomaceous Earth Ore to Reduce Organic Content

(75) Inventors: George A. Nyamekye, Snowflake, AZ (US); Timothy R. Smith, Lompoc, CA (US); Craig Smith, Santa Maria, CA (US); John Menear, Goleta, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: World Minerals, Inc., Santa Barbara, CA (US)

(21) Appl. No.: 12/439,595

(22) PCT Filed: Aug. 24, 2007
(86) PCT No.: PCT/US07/76749

§ 371 Date: Feb. 16, 2010

Related U.S. Application Data
(60) Provisional application No. 60/824,705, filed on Sep. 6, 2006.

(51) Int. Cl. B01J 20/02; A61L 2/04

(52) U.S. Cl. 502/412; 422/308

(57) ABSTRACT

Disclosed herein is a method for processing diatomaceous earth ore comprising roasting a diatomite feed at a temperature ranging from about 850 to about 1600° F. Also disclosed herein is a method for processing a diatomite feed comprising at least one impurity to produce a diatomite product having at least one of reduced loss-on-ignition, reduced impurity content, reduced beer soluble iron content, and improved brightness as compared to the feed composition. Further disclosed herein is a system for processing a diatomite feed comprising a first heating zone for roasting the diatomite at a temperature ranging from about 850 to about 1600° F., a second zone for calcining the diatomite at a temperature of at least about 1600° F., and a counter-current flow of gas from the second heating zone to the first heating zone.

### RELATED APPLICATION

This International PCT Application claims the right of priority to U.S. Provisional Patent Application No. 60/824,705, filed Sep. 6, 2006, which is incorporated by reference herein in its entirety.

### FIELD OF THE INVENTION

Disclosed herein is a method for processing diatomaceous earth ore comprising roasting a diatomite feed at a temperature ranging from about 850 to about 1600° F. Also disclosed herein is a method for processing a diatomite feed comprising at least one impurity to produce a diatomite product having at least one of reduced loss-on-ignition, reduced impurity content, reduced beer soluble iron content, and improved brightness as compared to the feed composition. Also disclosed herein is a system for processing a diatomite feed comprising a first heating zone for roasting the diatomite at a temperature ranging from about 850 to about 1600° F., a second zone for calcining the diatomite at a temperature of at least about 1600° F., and a counter-current flow of gas from the second heating zone to the first heating zone.

### BACKGROUND OF THE INVENTION

Diatomite is a sedimentary rock that comprises the fossilized skeletons of diatoms, which are unicellular aquatic plants related to algae. The skeletons comprise opal-like, amorphous silica (SiO₂.H₂O) containing small amounts of microcrystalline materials. Diatomite may also comprise water and small amounts of other substances such as Al₂O₃, Fe₂O₃, TiO₂, P₂O₅, CaO, and K₂O. The term diatomite, or diatomaceous earth, is inclusive of various diatom species that may occur in a wide variety of shapes, such as cylindrical, rod-like, and star-shaped diatoms.

Diatoms are generally characterized by a hollow interior and a perforated surface. Diatomite may take the form of siliceous frustules of diatoms, the majority of which have sizes ranging from 0.75 μm to 1,000 μm, such as from 10 μm to 150 μm. For instance, diatomite may have a particle size distribution ranging from 5 μm (d₁₀, defined as the size at which 10 percent of the particle volume is accounted for by particles having a diameter less than or equal to this value) to 82 μm (d₉₀, defined as the size at which 90 percent of the particle volume is accounted for by particles having a diameter less than or equal to this value).

The unique porous silica structure of diatomite may allow for high absorptive capacity and surface area, chemical stability, and low bulk density. These properties may make diatomite particularly useful for filtration processes, for example, in the food and beverage, biotechnology, pharmaceutical, and chemical industries. For instance, diatomite is often used to filter antibiotics, pharmaceuticals, chemicals, solvents, vitamins, edible oils and fats, fruit juices, glucose, sugar, water, beer, and wine. Diatomite may also be used as a filler in paints, paper, asphalt, and plastics.

Diatomite is obtained from the processing of diatomaceous earth ores. Diatomaceous earth ore may comprise up to 65% moisture and various organic and inorganic substances. Thus, before using diatomite in filtration processes, the raw material typically undergoes at least one conditioning process, such as drying, calcining, milling, crushing, grinding, grit separation, and/or water treatment. For instance, a diatomite feed may be dried and calcined to remove moisture, to convert organic substances into oxides, silicate, or aluminosilicates, and to sinter various undesirable inorganic compounds such as calcium carbonate, calcium sulfate, iron derivatives, and sulfides. Calcination may also serve to agglomerate the diatoms and their fragments into aggregates so as to reduce the content of fine particles and increase permeability.

Standard, or straight, calcination, which typically yields diatomite products that are pink in color, may be carried out at temperatures ranging from about 1100 to about 2400° F., such as from 1100 to 2200° F., from 1500 to 1800° F., or from 1600 to 2000° F. Diatomite may also be flux calcined by introducing an alkaline flux, for example a sodium compound such as sodium carbonate, during calcination. Flux calcination, which typically yields diatomite products that are white in color, may be performed to decrease the energy input required to calcine diatomite and/or to reduce the temperature at which sintering and agglomeration of diatomite particles occurs, thus permitting larger agglomerates. Flux calcination may be carried out at temperatures ranging from about 600 to about 2400° F., such as from 1500 to 2200° F. or from 1600 to 2000° F., or 1600 to 1800° F. During calcination, the porosity and specific surface area of the diatomite decrease and, in many cases, depending on the calcination process, at least a portion of the amorphous SiO₂ is transformed into a crystalline phase called cristobalite.

In contrast to calcining, it is also possible to heat the diatomite to lower temperatures and/or for shorter retention times such that many of the impurities are vaporized without, or without substantially, changing the other important physical properties of the natural diatomaceous earth. For instance, heating or roasting the diatomite feed may avoid the physical property changes that may be associated with calcining, such as increased agglomerate content, increased cristobalite content, and decreased specific surface area.

Diatomaceous earth deposits of commercial size are typically of marine or fresh water origin. High grade diatomaceous earth ore comprises hydrous silica and small concentrations of alumina, iron, and fluxing impurities such as alkaline earth metals. Diatomite obtained from these high grade deposits may have a relatively light color and low bulk density. However, these naturally occurring high quality deposits are limited in quantity. Lower grade diatomaceous earth ore may comprise higher amounts of organic substances as well as clay and carbonates. The higher content of organic matter and other impurities may impact the color of the diatomite obtained from these lower grade deposits. For instance, the lower grade diatomaceous earth ore may have any range of colors including light tan, gray, brown, greenish, and dark green.

Mineral deposits such as diatomite may be characterized by, among other things, their loss-on-ignition (LOI) value. In theory, the percentage weight lost by a sediment on ignition may give a crude measure of the organic content of the sediment. LOI may be determined by measuring weight loss in samples after burning at selected temperatures. The majority of diatomite's LOI may result from carbon oxidizing to carbon dioxide and the oxidation of sulfur to sulfur dioxide.

High grade diatomite products, for instance, fillers and filter aids, are often identified by their light color, high brightness, and/or low LOI value. Thus, lower grade crude ore, which tends to have a higher organic content and consequently higher LOI value, is not necessarily suitable for production of many typical diatomite products. Lower grade crude diatomaceous earth ore may also be less suitable for producing straight and flux calcined diatomite products. For example, during calcination, the presence of a high organic content may result in a reducing atmosphere in the kiln. Pink, or straight calcined, diatomite products tend to show high solubility under reducing conditions, which may lead to a lower quality product, for instance, a product having a higher beer soluble iron (BSI) content. Moreover, during flux calcination, the presence of a high organic content may result in the formation of black particles in the finished flux calcined product. These black particles tend to reduce the brightness of the diatomite products, which may be undesirable in the case of filler and filter aid products.

In addition, when the diatomite composition is used to filter goods for consumption, for instance liquids such as beer, additional considerations may become relevant. For example, in the case of beer, trace amounts of iron, referred to BSI, in the final beer product may shorten shelf life and impact beer flavor and color. Thus, one may seek to process the diatomite so as to keep the BSI content as low as possible.

The prior art discloses various preheating and calcination processes for minerals such as diatomite. For example, U.S. Pat. No. 5,710,090 describes a process to reduce cristobalite content during straight calcination of diatomite comprising calcining the diatomite at a temperature no higher than 850° C. U.S. Pat. No. 1,985,526 discloses a preheating treatment comprising subjecting diatomaceous earth to temperatures ranging from 900 to 1500° F., optionally followed by calcination at a temperature greater than 1500° F. Finally, JP 5-6037214 describes a preheating treatment process comprising heating a diatomite feed to a temperature greater than 300° C. prior to calcination. However, the prior art methods do not adequately address the problem of improving the brightness, color, BSI content, and/or LOI value of natural crude ore while still substantially maintaining the other important physical properties of the natural diatomaceous earth, such as low cristobalite content, low aggregate content, and higher specific surface area.

Thus, it would be useful to provide a method for processing diatomaceous earth ore to lighten its color, increase its brightness, decrease its BSI content, decrease its impurity content, and/or decrease its LOI value, without bringing about the physical property changes typically associated with calcination.

### BRIEF DESCRIPTION OF THE FIGURES

FIG. 1 is a process flow diagram illustrating a prior art system for processing diatomaceous earth.

FIG. 2 is a process flow diagram illustrating a system for processing diatomaceous earth in accordance with at least one embodiment of the present disclosure.

### SUMMARY OF THE INVENTION

Disclosed herein is a method for processing diatomaceous earth ore, or diatomite feed, comprising heating or roasting a diatomite feed having a loss-on-ignition of at least about 4% by weight at a temperature ranging from about 850 to about 1600° F. for a period sufficient to produce a diatomite product having a LOI value of less than or equal to about 2%.

Also disclosed herein is a method for processing diatomaceous earth ore comprising roasting a diatomite feed having a first LOI value and an impurity content of at least about 2% by weight at a temperature ranging from about 850 to about 1600° F. for a period sufficient to produce a diatomite product having a second LOI value and a reduced impurity content, wherein the second LOI value is at least about 50% less than the first LOI value.

Further disclosed herein is a method for processing diatomaceous earth ore comprising roasting a diatomite feed having a first brightness and an impurity content of at least about 2% at a temperature ranging from about 850 to about 1600° F. for a period sufficient to produce a diatomite product having a second brightness and a reduced impurity content, wherein the second brightness is at least about 5% brighter than the first brightness.

Still further disclosed herein is a method for processing diatomaceous earth ore comprising roasting a diatomite feed in a first heating zone at a temperature ranging from about 850 to about 1600° F., and calcining the diatomite feed in a second heating zone at a temperature greater than about 1600° F., wherein a counter-current flow of gas flows from the second heating zone to the first heating zone, and wherein the second heating zone has a higher oxygen content than the first heating zone.

Also disclosed herein is a system for processing diatomaceous earth ore comprising a first heating zone for roasting a diatomite feed at a temperature ranging from about 850 to about 1600° F., a second heating zone for calcining the diatomite feed at a temperature greater than about 1600° F., and a counter-current flow of gas flowing from the second heating zone to the first heating zone, wherein the second heating zone has a higher oxygen content than the first heating zone.

### DETAILED DESCRIPTION OF THE INVENTION

The present disclosure provides methods for processing diatomaceous earth ore, or diatomite feed, to provide a diatomite product suitable for use as high quality natural product. The processing method may include roasting a diatomite feed comprising at least one impurity in a suitable vessel or series of vessels at a temperature ranging from about 850 to about 1600° F., for a retention time sufficient to remove at least a portion of the at least one impurity. After roasting, the diatomite product may be used as high quality natural product, or may be optionally further processed.

In one embodiment, the processing method may comprise roasting a diatomite feed having a loss-on-ignition value of at least about 4% by weight at a temperature ranging from about 850 to about 1600° F. for a period sufficient to produce a diatomite product having a LOI value of less than or equal to about 2% by weight. In another embodiment, the processing method may comprise heating a diatomite feed having a first LOI value and an impurity content of at least about 2% by weight at a temperature ranging from about 850 to about 1600° F. for a period sufficient to produce a diatomite product having a second LOI value and a reduced impurity content, wherein the second LOI value is at least about 50% less than the first LOI value. In a further embodiment, the processing method may comprise heating a diatomite feed having a first brightness and an impurity content of at least about 2% at a temperature ranging from about 850 to about 1600° F. for a period sufficient to produce a product having a second brightness and a reduced impurity content, wherein the second brightness is at least about 5% brighter than the first brightness.

Suitable diatomite feed materials useful in the embodiments disclosed herein may be commercially available natural diatomite products, for example, Celite® S, Celite® 500, and Celite® NPP (World Minerals Inc., Santa Barbara, Calif.); and Celatom MN-2, MN-4, FN-1, FN-2, and FN-6 (EaglePicher Filtration & Minerals, Inc., Reno, Nev.). In addition, the methods of the present disclosure are applicable to any naturally occurring crude ore.

The diatomite feed composition may be characterized by various properties, for instance, brightness, impurity content, LOI value, BSI content, and cristobalite content. In at least one embodiment, the diatomite feed composition may have a blue light brightness (% BLB) ranging from about 20 to about 50. In certain embodiments, the % BLB may be less than about 50, less than about 40, or less than about 30. According to another embodiment, the diatomite feed composition may have a BSI content ranging from about 10 to about 50 mg Fe/kg product, for example, ranging from about 20 to about 40 mg Fe/kg product, or from about 20 to about 30 mg Fe/kg product. In yet another embodiment, the diatomite feed composition may have a cristobalite content of less than about 0.5% by weight, for example, less than about 0.4%, less than about 0.3%, or less than about 0.2% by weight. According to a further embodiment, the diatomite feed composition may have a LOI value of at least about 4%, for example, at least about 6%, at least about 8%, or at least about 10%. In still a further embodiment, the diatomite feed composition may have an impurity content of at least about 2% by weight relative to the total weight of the feed composition, for example, at least about 3%, or at least about 4% by weight.

As used herein, the term “impurity” is meant to encompass various undesirable organic or inorganic substances present in crude diatomaceous earth ore. For instance, diatomaceous earth ore may contain at least one impurity chosen from aluminum compounds, iron compounds, alkali and alkaline earth metal compounds, sulfur compounds such as sulfates and sulfides, clays, carbonates, and low levels of phosphate.

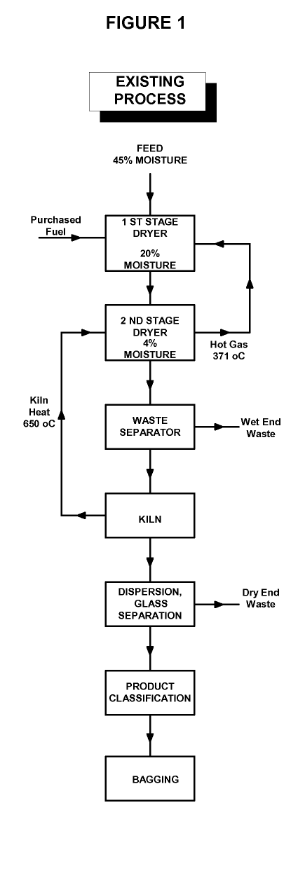

FIG. 1 is a process flow diagram illustrating a prior art system for processing diatomaceous earth. Conventionally, a diatomite feed comprising, for example, about 45% moisture, undergoes at least one drying stage, such as a first drying stage that may dry the diatomite to about 20% moisture and a second drying stage that may dry the diatomite to about 4% moisture. Subsequently, the dried feed may be introduced into a separator to remove wet end waste. The diatomite may then be calcined in a kiln or similar apparatus to remove various organic substances and to agglomerate the diatoms and their fragments. Following calcination, the calcined diatomite may undergo various optional processing steps, such as separation of grit and particles, product classification, and bagging.

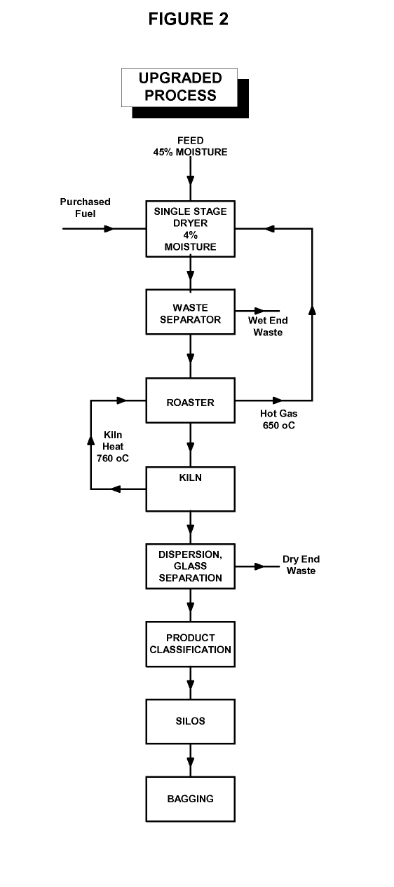

Referring now to FIG. 2, a system for carrying out at least one embodiment of the present disclosure is depicted. For instance, in FIG. 1, a diatomite feed comprising at least one impurity may be dried in a single stage dryer to a desired moisture level, for example, about 4% moisture. The dried diatomite may then be sent to a waste separator to remove any wet end waste. According to FIG. 2, the diatomite is then roasted in a suitable vessel or series of vessels at a temperature ranging from about 850 to about 1600° F., for a retention time sufficient to remove at least a portion of the at least one impurity. The at least one roasting vessel or first “zone” may be chosen from, for example, pre-heaters, flash heaters, flash calciners, flash roasting reactors, and toroidal fluid bed reactors. Examples of such vessels include flash calciners available from FFE Minerals, and the TORBED reactor available from Torftech Ltd. and discussed, for example, in U.S. Pat. No. 6,139,313. The vessel may be equipped with at least one means for heating the diatomaceous earth feed, for instance direct heating mechanisms such as internal hot air or gas flow, and indirect heating mechanisms utilizing external heat sources in combination with any heat transfer surface conventionally used in the art. In at least one embodiment, and as shown in FIG. 2, the at least one roasting vessel may be heated at least in part by a counter-current gas flow originating from another step in the process, for instance a subsequent calcination step or another process in the treatment plant. In another embodiment, at least part of the energy required to heat the feed may be obtained from the combustion of at least a portion of the organic content present in the diatomite feed.

In at least one embodiment, the feed may be roasted at a temperature ranging from about 850 to about 1600° F., for example, from about 900 to about 1000° F., or from about 1200 to about 1292° F. In another embodiment, the retention time may be less than about 4 minutes, for example, from about 2 to about 3 minutes, or from about 2 to about 10 seconds. While not wishing to be bound by theory, it is hypothesized that heating the ore at a lower temperature and/or for a shorter retention time serves to vaporize at least a portion of the impurities present in the diatomaceous earth while not producing the physical and/or structural changes that typically result from calcination (for example, increased cristobalite content, increased agglomerate content, and decreased specific surface area).

After processing, the resulting diatomite product may exhibit an increased brightness, decreased impurity content, and/or decreased LOI value. For instance, the diatomite product may have a brightness of at least about 5% greater than the brightness of the diatomite feed, for example at least about 10% or at least about 20% greater than the brightness of the diatomite feed. According to one embodiment, the diatomite product may have a % BLB of at least about 50, for example at least about 55 or at least about 60. In another embodiment, the diatomite product may have a BSI content of less than about 30 mg Fe/kg product, for example, ranging from about 20 to about 30 mg Fe/kg product, or from about 20 to about 25 mg Fe/kg product. According to yet another embodiment, the diatomite product may have a cristobalite content ranging from about 0.2% to about 0.5% by weight relative to the total weight of the diatomite product. For example, in certain embodiments, the cristobalite content may be less than about 0.5%, less than about 0.4%, less than about 0.3%, or less than about 0.2% by weight. In one embodiment, the cristobalite content of the diatomite product after roasting is not, or is not substantially, greater than the cristobalite content of the diatomite feed. In a further embodiment, the diatomite product may have a LOI value ranging from about 0.5% to about 2%. For example, in certain embodiments, the LOI value may be less than about 2%, less than about 1.5%, or less than about 1%. According to a still further embodiment, the diatomite product may have and impurity content of less than about 4% by weight relative to the total weight of the diatomite product, for example less than about 2% or less than about 1% by weight.

Following the roasting process, the diatomite product may be sold as high quality natural product or may be optionally further processed. For example, as shown in FIG. 2, the diatomite product may be transferred to a second vessel or series of vessels, or second “zone,” for further treatment. The means for transferring the diatomite product from the first zone to the second zone may include, for example, gravity flow. In at least one embodiment, the diatomite product may exit the first vessel or zone and enter the second vessel or zone by action of gravity. In another embodiment, the first zone and the second zone may exist within separate compartments of the same vessel. For instance, a roasting or calcining vessel may have separate compartments operating at different temperatures and with varying retention times.

The at least one second vessel may be any vessel suitable for the desired subsequent operation, for example, vessels suitable for grinding, milling, calcining, crushing, water treatment, and/or grit separation. In one embodiment, and as shown in FIG. 2, the at least one second vessel may be chosen from vessels suitable for calcining the diatomite product, such as rotary kilns and flash calciners. The at least one second vessel may be equipped with at least one means for heating the diatomite product, for instance direct heating mechanisms such as internal hot air or gas flow, and indirect heating mechanisms utilizing external heat sources in combination with any heat transfer surface conventionally used in the art.

While not wishing to be bound by theory, it is hypothesized that roasting the crude ore to remove at least a portion of impurities, such as organic content, may result in a more favorable (e.g., more oxidizing) calcination atmosphere in the second zone, which may lead to lower BSI content in the final calcined product. It is also hypothesized that the BSI content may be influenced by the calcination atmosphere. For example, it is believed that a more reducing calcination atmosphere may result in the reduction of any iron present in the diatomite to the Fe²⁺ state, which is a more soluble state. In contrast, it is believed that in a more oxidizing calcination atmosphere, any iron present in the diatomite remains in the Fe³⁺ state, which is less soluble. For instance, after roasting and calcining, the calcined diatomite product may have a BSI content ranging from 20 to 40 mg Fe/kg product, for instance less than about 40 mg Fe/kg or less than about 30 mg Fe/kg product.

The diatomite product may be calcined using any method known in the art, such as flux calcination and straight calcination. The calcination features, such as atmosphere, temperature, and retention time, may vary depending on the type of calcination, the equipment used, and/or the desired properties of the final calcined diatomite product.

The calcination may be performed in air, although other inert calcination environments may also be suitable. In one embodiment, the diatomite product may be calcined at a temperature of at least about 1600° F., for example, from about 1600 to about 2200° F., from about 1600 to about 2000° F., or from about 1600 to about 1800° F. Retention times may range from a few seconds to several minutes, for example, from about 2 to about 120 minutes. In another embodiment, the retention time may range from about 10 to about 40 minutes, or from about 30 to about 40 minutes.

In the case of flux calcination, at least one alkaline flux may be introduced during the calcination process. The alkaline flux may be chosen from any flux known in the art, for example, alkali metals such as sodium, potassium, rubidium, and cesium. The at least one alkali metal flux may be introduced in various forms, for example as alkali metal salts such as carbonates, chlorides, and nitrates. Examples of suitable alkali metal flux compounds include sodium carbonate, potassium carbonate, rubidium carbonate, and cesium carbonate. The at least one alkali metal compound may be present as flux during calcination in an amount ranging from 2 to 20% by weight, such as from 5 to 15%, or from 5 to 10% by weight, relative to the total weight of the diatomite product to be calcined.

Following completion of the calcination, the at least one alkali metal may be present in the flux calcined composition mostly in the form of at least one alkali metal oxide, such as Na₂O, K₂O, Rb₂O, and Cs₂O. The at least one alkali metal oxide may be present in the final flux calcined composition in an amount of at least 1% by weight, relative to the total weight of the flux calcined composition, for example the at least one alkali metal flux may be present in an amount ranging from 3 to 15% by weight, such as from 5 to 10% by weight.

Before and/or after calcination, the diatomite composition may be optionally subjected to further treatment, such as drying, crushing, milling, grinding, water treatment, and/or grit separation. For example, as shown in FIG. 2, the calcined diatomite may undergo grit and particle separation, product classification, storage, and bagging. In another embodiment, the calcined diatomite product may be further dried to remove moisture. Diatomite may be dried in flash and rotary dryers operating at temperatures ranging, for example, from 160 to 800° F. In certain embodiments, it may also be possible to lightly grind the diatomite before and/or after calcination to reduce the coarseness of the particles. In certain embodiments, the optional step of grinding is carried out so as to minimally increase the cake density and fine particle content of the diatomite composition. Excessive fine particles and high cake density may cause difficulty in filtering and undesirable turbidity during the filtering of liquids. In another embodiment, after calcination, the calcined diatomite product may be water treated to reduce the BSI content of the diatomite. In certain embodiments, increasing calcination time, such as calcining for at least one hour, and using water treatment may further reduce the BSI content.

When at least one optional additional processing step is included, for example, additional steps involving further heating and/or calcining the diatomite, it is possible to direct a gas flow from the at least one additional step performed, for example, in a second zone, to the roasting step performed, for example, in a first zone. For instance, as shown in FIG. 1, a gas flow from a subsequent calcination vessel may be directed into the roasting vessel. In at least one embodiment, the gas flow may be a counter-current gas flow. In another embodiment, the first zone may be heated at least in part by a counter-current gas flow from the second zone. In yet another embodiment, the second zone may have a higher oxygen content than the first heating zone. In theory, the presence of organic content in the feed composition leads to a more reducing atmosphere during heating and/or calcination. Thus, although not wishing to be bound by theory, in the present disclosure the second, or subsequent, zone has a higher oxygen content due to the vaporization of at least a portion of the organic content of the feed composition in the first zone, thus allowing for a more oxidizing atmosphere in the second zone.

Cristobalite content may be measured, for example, by the quantitative X-ray diffraction method outlined in H. P. Klug and L. E. Alexander, X-Ray Diffraction Procedures for Polycrystalline and Amorphous Materials, 2^{nd} ed., John Wiley & Sons: New York, pp. 531-563 (1972). According to this method, a sample is milled in a mortar and pestle to a fine powder, then back-loaded into a sample holder. The sample and its holder are placed into the beam path of an X-ray diffraction system and exposed to collimated X-rays using an accelerating voltage of 40 kV and a current of 20 mA focused on a copper target. Diffraction data are acquired by step-scanning over the angular region representing the interplanar spacing within the crystalline lattice structure of cristobalite that yields the greatest diffracted intensity. This region ranges from 21 to 23 2θ°, with data collected in 0.05 2θ° steps, counted for 20 seconds per step. The net integrated peak intensity is compared with those of standards of cristobalite prepared by the standard additions method in amorphous silica to determine the weight percent of the cristobalite phase in a sample.

The solubility of iron from diatomite products in beer (BSI content) may be determined using a calibrated colorimetric method employing 1,10-phenanthroline (i.e., o-phenanthroline, C₁₂H₈N₂). For example, a 5 g sample of diatomite is added to 200 mL of decarbonated beer (for example, BUDWEISER, registered trademark of Anheuser-Busch) at room temperature, and the mixture is swirled intermittently for an elapsed time of 5 min and 50 sec. The mixture is then immediately transferred to a funnel containing 25 cm diameter filter paper, from which the filtrate collected during the first 30 sec is discarded. Filtrate is collected for the next 150 sec, and a 25 mL portion is treated with approximately 25 mg of ascorbic acid (i.e., C₆H₈O₆), to reduce dissolved iron ions to the ferrous (i.e., Fe²⁺) state (thus yielding a “sample extract”). The color is developed by addition of 1 mL of 0.3% (w/v) 1,10-phenanthroline, and, after 30 min, the absorbance of the resulting sample solution is compared to a standard calibration curve. The calibration curve is prepared from standard iron solutions of known concentration in beer. Untreated filtrate is used as a method blank to correct for turbidity and color. Absorbance is measured at 505 nm using a spectrophotometer.

The loss-on-ignition value may be determined by various methods, for example, by placing a 100 gram sample of diatomite in a crucible, heating it in a muffle furnace to temperature of 1000° C. for one hour, and weighing the sample before and after heating. The percentage of mass lost after heating may be calculated as the loss-on-ignition.

The blue light brightness of samples may be measured, for example, using calculations derived from Hunter scale color data collected on a Spectro/plus Spectrophotometer (Color and Appearance Technology, Inc., Princeton, N.J.). A krypton-filled incandescent lamp is used as the light source. The instrument is calibrated according to the manufacturer's instructions using a highly polished black glass standard and a factory calibrated white opal glass standard. A plastic plate having a depressing machined into it is filled with sample, which is then compressed with a smooth-faced plate using a circular pressing motion. The smooth-faced plate is carefully removed to ensure an even, unmarred surface. The sample is then placed under the instrument's sample aperture for the measurements. The blue light brightness can then be calculated from the Hunter scale coordinates, L*, a*, and b*, according to the formula given below:

$$\% B L B = 0.01 {L}^{* 2} - \frac{( {L}^{*} ) ( {b}^{*} )}{70}$$

With the Hunter L*a*b* coordinates, components a, b, and L are the color component values on the color space scale as measured by a Hunter Ultrascan XE instrument. “+a” is a measure of red tint; “−a” is a measure of green tint; “+b” is a measure of yellow tint; “−b” is a measure of blue tint; “L” is a measure of whiteness.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, examples of certain embodiments of the present disclosure are given below.

### EXAMPLES

### Example 1

Various samples of diatomaceous earth ore having a particle size such that 100% of the particles pass through 150 mesh, a bulk density of 10 lb/ft³, and up to 11% LOI were calcined before and after roasting the diatomite in accordance with the present disclosure. For the calcination, 5 lb of feed was added to a rotary batch calciner that had been heated up to 2000° F. by the combustion of natural gas. The sample was held inside the calciner for a period of 20 minutes as it rotated at 4 revolutions per minute. For the roasting process, the roaster reactor was heated up to 1292° F. A dried feed material was introduced to the bottom of the vessel. The roasting temperature was maintained at this level with a retention time of less than 2 minutes. The hydrocarbons in the feed material were combusted by the time it exhausted through the top of the vessel. The roasted solids were collected using a high efficiency cyclone. The BSI content of each sample was measured. The results are shown below in Table 1.

TABLE 1

| | BSI - Calcining without | BSI - Roasting then |
|---|---|---|
| | Roasting | Calcining |
| | (mg Fe/kg Product) | (mg Fe/kg Product) |
| Sample 1 | 44.2 | 22.5 |
| Sample 2 | 64.9 | 22.1 |
| Sample 3 | 53.6 | 24.5 |
| Sample 4 | 51.6 | 22.2 |
| Sample 5 | 45.9 | 25.1 |
| Sample 6 | 43.8 | 24.1 |
| Sample 7 | 42.4 | 23.8 |
| Sample 8 | 43.7 | 24.0 |

As shown in Table 1 above, roasting diatomaceous earth ore in accordance with the present disclosure prior to calcination results in a reduction in the BSI content. For example, the treated diatomite may exhibit at least about a 40% reduction in BSI content as compared to the untreated diatomaceous earth.

### Example 2

A crude diatomaceous earth ore having a LOI value of 11.52% and a cristobalite content of 0.43% by weight was fed at a rate of 12 kg/h into a flash roasting reactor operating at 725° C. (1337° F.). The retention time of the feed in the reactor ranged from about 1 to 2 minutes. The roasted diatomite product had a LOI value of 1.67% and a cristobalite content of 0.37% by weight.

### Example 3

Example 2 was repeated using a feed rate of 24 kg/h and a reactor temperature of 700° C. (1292° F.). The roasted diatomite product had a LOI value of 1.91% and a cristobalite content of 0.44% by weight.

### Example 4

Example 3 was repeated using a reactor temperature of 725° C. (1337° F.). The roasted diatomite product had a LOI value of 1.69% and a cristobalite content of 0.43% by weight.

### Example 5

Example 3 was repeated using a reactor temperature of 750° C. (1382° F.). The roasted diatomite product had a LOI value of 1.65% and a cristobalite content of 0.46% by weight.

### Example 6

Example 3 was repeated using a reactor temperature of 800° C. (1472° F.). The roasted diatomite product had a LOI value of 1.34% and a cristobalite content of 0.46% by weight.

### Example 7

Example 3 was repeated using a reactor temperature of 825° C. (1517° F.). The roasted diatomite product had a LOI value of 1.08% and a cristobalite content of 0.41% by weight.

### Example 8

Example 3 was repeated using a reactor temperature of 850° C. (1562° F.). The roasted diatomite product had a LOI value of 1.00% and a cristobalite content of 0.40% by weight.

### Example 9

Example 1 was repeated using a feed rate of 48 kg/h. The roasted diatomite product had a LOI value of 1.56% and a cristobalite content of 0.46% by weight.

The results obtained from Examples 2-9 are illustrated in Table 2 below. The LOI and cristobalite values for untreated feed and bright ore are included for purposes of comparison.

TABLE 2

| | Feed Rate | Temperature | | Cristobalite |
|---|---|---|---|---|
| | (kg/h) | (° C.) | LOI (%) | Content (wt %) |
| Untreated Feed | — | — | 11.52 | 0.43 |
| Example 2 | 12 | 725 | 1.67 | 0.37 |
| Example 3 | 24 | 700 | 1.91 | 0.44 |
| Example 4 | 24 | 725 | 1.69 | 0.43 |
| Example 5 | 24 | 750 | 1.65 | 0.46 |
| Example 6 | 24 | 800 | 1.34 | 0.46 |
| Example 7 | 24 | 825 | 1.08 | 0.41 |
| Example 8 | 24 | 850 | 1.00 | 0.40 |
| Example 9 | 48 | 725 | 1.56 | 0.46 |
| Bright Ore | — | — | 4 to 6 | 0.47 |

As shown in Table 2 above, roasting diatomaceous earth ore in accordance with the present disclosure results in a reduction in the LOI value, without a substantial corresponding increase in cristobalite content. For example, the LOI value of the treated diatomite product may be as low as about 9% of the original LOI value (see, e.g., Example 8), while the cristobalite content does not, or does not substantially, increase.

In addition, the chemical makeup of the untreated feed and treated diatomite products are illustrated below in Table 3 for purposes of comparison:

TABLE 3

| | Feed | | | | | | | | | | | | |
|---|---|---|---|---|---|---|---|---|---|---|---|---|---|
| | Rate | Temp. | | | | | | | | | | | |
| | (kg/h) | (° C.) | Na₂O | MgO | Al₂O₃ | SiO₂ | P₂O₅ | S | Cl | K₂O | CaO | TiO₂ | Fe₂O₃ |
| Untreated | — | — | 0.54 | 0.61 | 4.17 | 91.0 | 0.26 | 1.20 | 0.04 | 0.71 | 0.58 | 0.22 | 1.77 |
| Feed | | | | | | | | | | | | | |
| Example 2 | 12 | 725 | 0.54 | 0.59 | 4.25 | 90.7 | 0.25 | 0.13 | 0.05 | 0.76 | 0.65 | 0.23 | 1.68 |
| Example 3 | 24 | 700 | 0.54 | 0.62 | 4.48 | 90.4 | 0.26 | 0.17 | 0.03 | 0.81 | 0.71 | 0.24 | 1.73 |
| Example 4 | 24 | 725 | 0.54 | 0.59 | 4.46 | 90.4 | 0.25 | 0.15 | 0.04 | 0.77 | 0.68 | 0.23 | 1.66 |
| Example 5 | 24 | 750 | 0.55 | 0.60 | 4.28 | 90.7 | 0.25 | 0.08 | 0.05 | 0.75 | 0.62 | 0.23 | 1.71 |
| Example 6 | 24 | 800 | 0.53 | 0.63 | 4.43 | 90.4 | 0.26 | 0.10 | 0.04 | 0.77 | 0.65 | 0.23 | 1.77 |
| Example 7 | 24 | 825 | 0.47 | 0.60 | 4.11 | 91.1 | 0.24 | 0.09 | 0.03 | 0.71 | 0.58 | 0.22 | 1.68 |
| Example 8 | 24 | 850 | 0.49 | 0.61 | 4.13 | 91.0 | 0.25 | 0.08 | 0.03 | 0.72 | 0.59 | 0.22 | 1.70 |
| Example 9 | 48 | 725 | 0.59 | 0.64 | 4.75 | 89.7 | 0.28 | 0.10 | 0.04 | 0.86 | 0.77 | 0.26 | 1.84 |

As shown in Table 3 above, in all instances, roasting the diatomaceous earth ore in accordance with the present disclosure reduces the sulfur content of the diatomite product as compared to the untreated feed.

### Example 10

Crude diatomaceous earth ore having a blue light brightness (% BLB) of 32.2 was roasted at a temperature of 650° C. (1202° F.) in a roaster reactor for a retention not exceeding 60 seconds at a feed rate of 50 kg/hr. Combustion was controlled to obtain flue gas that had no more than 3% oxygen concentration. The roasted diatomite product had a % BLB of 53.9.

### Example 11

The feed material of Example 10 was roasted at a temperature of 700° C. (1292° F.) in the roaster reactor for a retention not exceeding 120 seconds at a feed rate of 50 kg/hr. Combustion was controlled to obtain flue gas that had no more than 7% oxygen concentration. The roasted diatomite product had a % BLB of 52.7.

### Example 12

The feed material of Example 10 was roasted at a temperature of 675° C. (1247° F.) in the roaster reactor for a retention not exceeding 60 seconds at a feed rate of 50 kg/hr. Combustion was controlled to obtain flue gas that had no more than 5% oxygen concentration. The roasted diatomite product had a % BLB of 53.1.

The results obtained from Examples 10-12 are illustrated in Table 4 below. The values for untreated feed and bright ore, are included for purposes of comparison.

TABLE 4

| | L | a | B | % BLB |
|---|---|---|---|---|
| Feed | 64.52 | 0.99 | 10.2 | 32.2 |
| Example 10 | 78.08 | 0.94 | 6.31 | 53.9 |
| Example 11 | 77.51 | 1.11 | 6.63 | 52.7 |
| Example 12 | 77.71 | 1.01 | 6.55 | 53.1 |
| Bright Ore | 80.07 | 1.34 | 7.87 | 55.1 |

As shown in Table 4 above, roasting diatomaceous earth ore in accordance with the present disclosure results in an increase in brightness. For example, the brightness of the treated diatomite product may be up to 67% greater than the brightness of the feed ore (see, e.g., Example 10). Moreover, the treated diatomite product has a brightness more nearly resembling that of natural high quality ore.

1-54. (canceled)

55. A method for processing diatomite comprising roasting a diatomite feed in a first heating zone at a temperature ranging from about 850° F. to about 1600° F. to produce a diatomite product, and calcining the diatomite product in a second heating zone at a temperature greater than about 1600° F. to produce a calcined diatomite product,
wherein a counter-current flow of gas flows from the second heating zone to the first heating zone, and
wherein the second heating zone has a higher oxygen content than the first heating zone.

56. The method of claim 55, wherein the diatomite feed is roasted at a temperature ranging from about 900° F. to about 1000° F.

57. The method of claim 55, wherein the diatomite feed is roasted for a retention time of at least about 4 minutes.

58. The method of claim 55, wherein the diatomite product is calcined at a temperature ranging from about 1600 to about 2400° F.

59. The method of claim 55, wherein the diatomite product is calcined for a retention time ranging from about 2 to about 120 minutes.

60. The method of claim 55, wherein the diatomite product is flux calcined in the presence of at least one alkali metal flux.

61. The method of claim 55, wherein the first heating zone is heated at least in part by the counter-current flow of gas.

62. A system for processing diatomaceous earth ore comprising a first heating zone for roasting a diatomite feed at a temperature ranging from about 850° F. to about 1600° F. to produce a diatomite product, a second heating zone for calcining the diatomite product at a temperature greater than about 1600° F. to produce a calcined diatomite product, and a counter-current flow of gas flowing from the second heating zone to the first heating zone,
wherein the second heating zone has a higher oxygen content than the first heating zone.

63. The system of claim 62, further comprising at least one means for transferring the diatomite product from the first heating zone to the second heating zone.

64. The system of claim 62, wherein the first heating zone is heated at least in part by the counter-current flow of gas.

65. A method for processing diatomite, comprising roasting a diatomite feed having a loss-on-ignition of at least about 4% by weight at a temperature ranging from about 850° F. to about 1600° F. for a retention time sufficient to produce a diatomite product having a loss-on-ignition value of less than or equal to about 2% by weight.

66. The method of claim 65, wherein the diatomite product has a loss-on-ignition value of less than or equal to about 1% by weight.

67. The method of claim 65, wherein the diatomite product has a loss-on-ignition value ranging from about 0.5% to about 2% by weight.

68. The method of claim 65, wherein the diatomite product has a cristobalite content of less than about 0.5% by weight.

69. The method of claim 65, wherein the retention time is less than about 4 minutes.

70. The method of claim 65, wherein the retention time ranges from about seconds to about 120 seconds.

71. The method of claim 65, wherein the diatomite feed is roasted at a temperature ranging from about 900° F. to about 1000° F.

72. The method of claim 65, further comprising subjecting the diatomite feed to at least one treatment chosen from the group consisting of calcining, drying, crushing, milling, grinding, grit separation, and water treatment.

73. The method of claim 65, further comprising subjecting the diatomite product to at least one treatment chosen from the group consisting of calcining, drying, crushing, milling, grinding, grit separation, and water treatment.

74. A method for processing diatomite comprising roasting a diatomite feed having a first loss-on-ignition value and an impurity content of at least about 2% by weight at a temperature ranging from about 850° F. to about 1600° F. for a retention time sufficient to produce a diatomite product having a second loss-on-ignition value and a reduced impurity content,
wherein the second loss-on-ignition value is at least about 50% less than the first loss-on-ignition value.
